(19) United States
(12) Patent Application Publication
(10) Pub. No.: US 2010/0063472 A1
(43) Pub. Date: Mar. 11, 2010

(54) COMPOSITIONS AND METHODS FOR IMPROVED OCCLUSION OF VASCULAR DEFECTS

(76) Inventors: TIMOTHY A. BECKER, Ann Arbor, MI (US); Daryl R. Kipke, Dexter, MI (US); Cameron G. McDougall, Phoenix, AZ (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 12/436,641

(22) Filed: May 6, 2009

Related U.S. Application Data
(63) Continuation of application No. 10/738,317, filed on Dec. 17, 2003, now abandoned.

(51) Int. Cl. A61M 31/00

(52) U.S. Cl. 604/507

(57) ABSTRACT

The present invention comprises compositions and methods for forming an endovascular occlusion to treat conditions such as aneurysms, arterio-venous malformations, excessive blood supply to tumors, massive vascular hemorrhaging, and other conditions which require an embolization to alleviate the condition. Embodiments of the present invention comprise compositions and methods that use calcium alginate, without or without endovascular coils or similar devices, to form occlusions at a site within the mammalian body targeted for occlusion.

### FIELD OF THE INVENTION

The present invention relates generally to compositions and methods for forming an endovascular occlusion to treat conditions such as aneurysms, arterio-venous malformations, excessive blood supply to tumors, massive vascular hemorrhaging, and other conditions which require an embolization to alleviate the condition. More particularly, the present invention relates to compositions and methods that use calcium alginate, without or without endovascular coils or similar devices, to fowl occlusions at a site within the mammalian body targeted for occlusion.

### BACKGROUND OF THE INVENTION

Neurovascular lesions and cerebral tumors threaten the lives of millions throughout the world. Aneurysms, arteriovenous malformations (“AVMs”), and tumors in the brain affect a wide range of patient ages and ethnicities. The frequency of lesion growth is spread evenly across all ethnic groups.

Aneurysms often form over time from a genetic defect in the elastic development of a blood vessel. Normal pressures eventually stress the wall, slowly forming a balloon on the side of the vessel wall (aneurysm sac). Typically, patients develop aneurysms slowly over time and are of high risk to people over 40. However, hemorrhage and other complications can occur as early as age 20. Presently 160,000 aneurysm patients are diagnosed annually (40,000 in the North America, 120,000 in Europe) because of vessel hemorrhage. After hemorrhage, only 60% of these patients will survive.

AVMs are known to be congenital defects that grow readily in the first ten years of life. Approximately 2 million people in North America and Europe have AVMs. High blood flows begin to shunt through the AVM, thereby expanding and weakening the vessel lesion over time. About 7% of AVM patients in North America alone undergo vessel weakening and hemorrhage. AVM hemorrhages generally affect children and young adults between the ages of 20 and 40.

As discussed in U.S. Pat. No. 6,592,566, which is hereby incorporated by reference, endovascular polymer treatment is a new and growing field for achieving vascular occlusion of blood flow and treating affected groups. With this technique, polymer materials may be injected directly into blood vessels so that the polymer material will travel to the targeted site in the vascular system and polymerize to form an endovascular occlusion at the target site.

Endovascular embolization techniques have grown with advances in catheter technologies over the past five years. Microcatheters facilitate greater access to previously unreachable vascular lesions.

Aneurysms that are unreachable by surgical means are currently treated with endovascular metal coils, with limited success. Coils are often platinum-based shape-memory wires that are fed into the aneurysm from a microcatheter. As the coils are released from the catheter tip, they are packed into the aneurysm space. Coils are an improvement over invasive surgical techniques and provide an alternative to previously untreatable lesions. However, endovascular coils have significant limitations as well. They are difficult to control during placement, and they can become tangled or protrude into the blood flow stream, increasing the likelihood of clot formation and stroke. Moreover, coils can fill only about 30% of the volume of an aneurysm. The coil mass can therefore compact on itself over time, allowing the aneurysm to continue growing.

Thus a need remains for compositions and methods that use suitable biological materials, with or without endovascular coils or similar devices, to effectively form therapeutic occlusions at targeted sites within the mammalian body.

### BRIEF SUMMARY OF THE INVENTION

The present invention addresses this unmet need by providing compositions and methods that use calcium alginate, with or without endovascular coils or similar devices, to form occlusions at a site within the mammalian body targeted for occlusion. Thus, in accordance with the present inventions, beneficial use of a non-adhesive, non-toxic, and tissue-like material, such as calcium alginate, can expand endovascular embolization to fill the need for greater therapeutic effectiveness and minimized risk, and endovascular embolization can be a more effective substitute or adjunct to more invasive surgery and radiosurgery techniques.

The present invention is comprised of a novel treatment method for endovascular occlusion that optimizes alginate with various microcatheter delivery systems. In accordance with some embodiments of the invention, alginate embolization materials are used with coils for aneurysm treatment, as well as treatments for AVMs and blood supplies to tumors.

In some embodiments of the inventions, calcium alginate is selectively delivered as a two-component polymer to blood vessels from microcatheters to produce effective endovascular polymer occlusion. The flow properties and the viscosity of liquid alginate can be used to optimize its delivery through microcatheters. Moreover, in some embodiments, a large volume of alginate may be delivered from microcatheters to the vessel system for a more complete occlusion without the concern of the catheter being glued to the vessel wall.

In some embodiments, injection of alginate and of its separate reactive components allows multiple options for endovascular occlusion. Current endovascular polymers are pre-mixed with a catalyst and polymerize within a specific time. The polymerization is irreversible, and the polymer attaches to the vessel, blocks the lumen of the injection catheter, and sometimes can due the catheter tip to the vessel wall. Embodiments of the invention comprise a non-adhesive alginate gel that provides greater flexibility and control of the polymerization process over current endovascular embolization materials.

In some embodiments, the invention comprises systems and methods to effectively occlude small-neck, low-flow aneurysms. Alternatively, in some embodiments, the invention comprises systems and methods to reduce potential outflow of wide-neck, high-flow aneurysms, for example, with assist devices, such as the combination of alginate with coils, to provide a treatment solution for these aneurysms.

### BRIEF DESCRIPTION OF THE DRAWINGS

The features and inventive aspects of the present invention will become more apparent upon reading the following detailed description, claims, and drawings, of which the following is a brief description:

FIG. 1(a) is a drawing of alginate structure.

FIG. 1(b) is a representation of alginate reaction upon application of calcium ions.

FIG. 2 is a flow diagram summary of alginate, coil, stent, and balloon occlusion options.

FIG. 3 is a diagram of a concentric tube catheter design for improved control of alginate injection.

FIG. 4(a) is a diagram showing alginate mass formation with a concentric tube catheter.

FIG. 4(b) is a diagram showing release of alginate the resulting mass from the concentric tube.

FIG. 5 is a diagram showing stent placement and alginate injection to completely fill an aneurysm.

FIG. 6 is a diagram showing partial aneurysm filling with coils, complete filling of remaining volume with alginate.

FIG. 7(a) is a photograph depicting an ALGEL-coated coil with a 3× diameter increase.

FIG. 7(b) is a photograph depicting a dehydrated coil at 1.08×.

FIG. 7(c) is a photograph depicting an ALGEL-coated coil rehydrated for 5 minutes at 1.7×.

FIG. 8(a) is a chart of viscosity versus concentration of various alginate molecular weights (apparent viscosities).

FIG. 8(b) is a chart of alginate strength and polymer yield versus various alginate molecular weights (apparent viscosities).

FIG. 9 is a drawing of an in vitro vessel cast aneurysm model setup.

FIG. 10(a) is a photograph showing a pre-embolization of a small-neck aneurysm.

FIG. 10(b) is a photograph showing coil delivery with partial aneurysm filling, <%5 of vol.

FIG. 10(c) is a photograph showing alginate filling of remaining aneurysm volume, 90-100% of vol.

FIG. 10(d) is a photograph showing post-embolization with complete aneurysm filling.

FIG. 11(a) is a photograph showing a pre-embolism stage of a wide-neck aneurysm.

FIG. 11(b) show addition of unmodified coils and alginate

FIG. 11(c) is a photograph showing a post-embolization stage with complete occlusion.

FIG. 12 is a chart of mechanical stability and fatigue resistance of high and low molecular weight alginate over 2 weeks in an in vitro aneurysm model.

FIG. 13 is a representation of a swine rete mirabile structure and anastomosis procedure.

FIG. 14 is a photograph of flow immediately after occlusion. Flow in the AP vessel is stopped, yet the AA and RA vessels maintain flow to the RM and CW.

FIG. 15 is the alginate occlusion of the AP vessel sustained after 6 months. Image shows signs of angiogenesis, a new vessel has formed to feed the base of the RM.

FIG. 16(a) is a photograph of pre-embolization of in vivo aneurysm model.

FIG. 16(b) is a photograph of alginate occlusion with balloon protection to completely fill the aneurysm sac

FIG. 16(c) is a photograph of post-embolization, complete occlusion of aneurysm with no parent vessel occlusion

FIG. 17 is histology of an alginate occlusion in the RM after six months. Tissue encapsulation and endothelial growth surrounds and penetrates the gel.

### DETAILED DESCRIPTION OF THE INVENTION

The present invention comprises compositions and methods that use calcium alginate, with or without endovascular coils, stents, balloons, or similar devices, to fowl occlusions at or within a site within the mammalian body targeted for occlusion.

In some embodiments of the inventions, calcium alginate, a biocompatible and mechanically stable two-component polymer, is selectively delivered as a two-component polymer to blood vessels from microcatheters to produce effective endovascular polymer occlusion. Purified calcium alginate has optimal material characteristics for use as an endovascular embolic agent. Alginate has an adjustable viscosity in its liquid form, mechanical stability in its solid form, and non-adhesive properties. The flow properties and the viscosity of liquid alginate can be used to optimize its delivery through microcatheters.

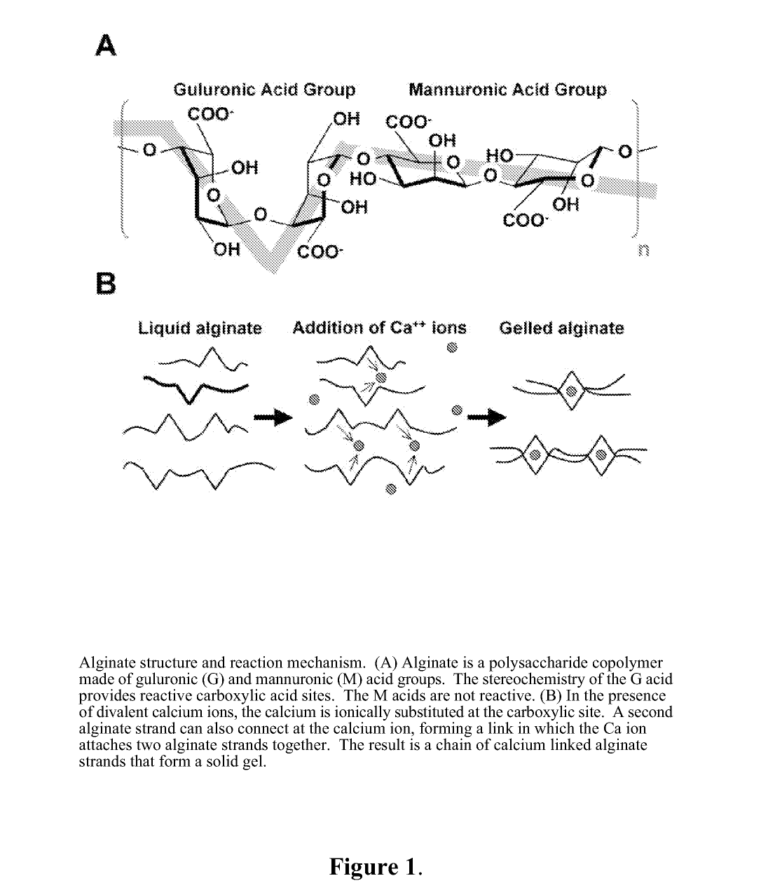

Alginic acid is a natural polysaccharide gel derived from brown algae. Alginate is a co-polymer consisting of blocks of mannuronic (M) and guluronic (G) acids in various arrangements along the polymer chain (FIG. 1(A)) and in various molecular weights. The concentration of G and M acids (the G/M ratio) contributes to varied structural and biocompatibility characteristics. Alginic acid is soluble in water and can be ionically cross-linked with a non-toxic divalent cation solution, such as calcium chloride (FIG. 1(B)). The calcium ions bind the guluronic acid sites of individual alginate molecules together to form a stable alginate gel. The resulting polymer has non-adhesive, tissue-like mechanical properties. Purified alginates with a high G acid content (PHG) have optimal material properties for use in endovascular occlusion.

Thus, calcium alginate is a natural polymer with a simple structure and high water content, allowing diffusion of the reactive component, calcium chloride, and biological fluids into the polymer. In particular, PHG alginate is biocompatible, requires no harsh solvents, and is non-adhesive.

In some embodiments, a large volume of alginate may be delivered from microcatheters to the vessel system for a more complete occlusion without the concern of the catheter being glued to the vessel wall. As one example, without limitation, a dual-lumen catheter can be used to inject the alginate and the calcium chloride reactive component simultaneously, allowing for flow direction of the polymer to the vessels requiring occlusion. Also, multiple catheters can be used to inject the alginate and reactive components from opposite directions (bi-directional injection) and allow the flows to meet and polymerize. Other feasible injection techniques include local flow arrest with a proximal balloon catheter and distal retrograde injection of alginate and the reactive component.

Alginate and its separate reactive components may allow for multiple options for endovascular occlusion. Current endovascular polymers are pre-mixed with a catalyst and polymerize within a specific time. The polymerization is irreversible, and the polymer attaches to the vessel, blocks the lumen of the injection catheter, and sometimes can glue the catheter tip to the vessel wall. The non-adhesive alginate gel may provide greater flexibility and control of the polymerization process over current endovascular embolization materials.

Material injectability and mechanical characterization are important for selecting a suitable aneurysm occlusion polymer, yet few have been extensively investigated. Our studies have shown that calcium alginate, as one example only and without limitation, ALGEL® (Neural Intervention Technologies, Ann Arbor, Mich.), is a non-adhesive material with high mechanical strength in its reacted solid four', low viscosity in its unreacted liquid form, and controllability during injection.

Our investigation shows that ALGEL alone can effectively occlude small-neck, low-flow aneurysms. However, wide-neck, high-flow aneurysms require assist devices to reduce potential outflow. According to the invention, ALGEL combined with coils is an effective treatment solution for these aneurysms, and controlled ALGEL delivery can eliminate flow to aneurysms and, when combined with coils or other devices, can eliminate the potential for ALGEL outflow from wide-neck, high flow aneurysms.

Without limitation, in one embodiment, the invention comprises controlled injection of alginate to the target site using a concentric tube microcatheter delivery system. In another embodiment, the invention comprises insertion of unmodified coils with or without stent placement, at the targeted site, followed by alginate injection. In yet another embodiment, the invention comprises insertion of modified coils, with or without stent placement, at the targeted site, followed by alginate injection. In another embodiment, the invention comprises insertion of modified alginate coated coils, with or without stent placement, at the targeted site.

In some embodiments, the claimed invention is comprised of an in vitro aneurysm model, which allows testing of some embodiments. The model provides flexible design and easy access for occlusion cast removal to expedite material testing of alginate and alginate-coil embolization for mechanical stability and fatigue resistance. The model permits identification of the polymer plug, as well as tracking any potential downstream embolus. Using the model, alginate flows may be tracked, for example, using a radioopaque dye so that any alginate flow can be recorded on the angiogram imaging system during injection, or by equipping the model's outflow paths with narrow outlet connectors (less than lumen diameter) to catch any potential alginate particles that are released downstream. In the invention, alginate particles can be read immediately by the real-time pressure readings that are taken at the model's two outlets. The outlet that becomes clogged will have a significant pressure drop (simulating a stroke). The pressure reading at the second outlet will also rise significantly due to compensation for lost flow.

The invention is also comprised of methods and compositions to enhance treatment options with a variety of occlusion techniques, ranging from alginate delivery systems to modified alginate-coil systems. Alginate is a highly biocompatible material with desirable characteristics for filling and occluding vessel lesions. Its unique material properties can be utilized independently or in combination with endovascular coils or other devices to maximize vessel occlusion and enhance the short- and long-term alginate embolization characteristics. Polymer embolization offers a significant complement and advantage to coiling alone. Thus, in accordance with the inventions, ALGEL's effectiveness as an occlusion material alone and in combination with other devices can increase application to a variety of neurovascular lesions, such as AVMs, aneurysms, and tumors.

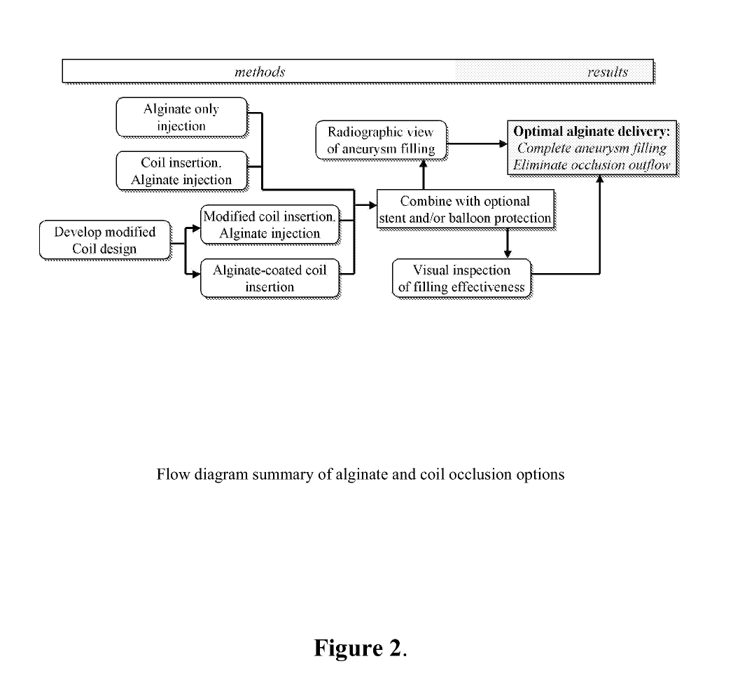

Embodiments of the invention may comprise, without limitation (FIG. 2):
Controlled injection of alginate using a concentric tube or dual lumen microcatheter delivery system;
Catheter placement to deliver alginate, with balloon inflated across the aneurysm neck;
Insertion of unmodified coils with or without stent and/or balloon placement, followed by alginate injection;
Insertion of modified coils, with or without stent and/or balloon placement, followed by alginate injection;
Insertion of modified alginate coated coils, with or without stent and/or balloon placement.

Currently, coil technology is useful to disrupt flow into the aneurysm and help activate thrombus formation within an aneurysm. However, due to the nature of coil delivery and the potential for entanglement during treatment, coils can fill only 25% to 33% of the aneurysm fundus space. The remaining space is filled by a thrombus. Continued pulsatile blood pressure on the aneurysm can force the coils to compact. The thrombus provides no mechanical strength to prevent this occurrence. The aneurysm can therefore continue to grow, and the risk of hemorrhage returns. In accordance with some embodiments of the inventions, combining coils with alginate can ensure more complete filling of the aneurysm, increase control of delivery, and decrease the potential for occlusion failure or polymer outflow into the blood stream.

In some embodiments, the invention is comprised of a modified coil coated with a calcium ion releasing material. The coil obstructs flow into the body of the aneurysm (fundus) and the fundus becomes diffused with calcium ions. Alginate is then injected into the targeted site, as one example only and without limitation, from a single-lumen microcatheter, to fill the remaining space.

In other embodiments, the invention is comprised of modified coil with a dehydrated alginate coating. When applied at the target site, the coil's alginate hydrogel rehydrates and swells to fill the aneurysm fundus.

### Optimal Alginate Delivery

Some embodiments of the invention comprise novel occlusion materials and delivery methods. Aneurysms are high-risk lesions that require precise delivery of treatment materials to avoid aneurysm rupture due to overfilling or embolus flowing upstream and causing a stroke. Neuroradiologists can accurately assess the treatment risk by analyzing factors such as aneurysm size, shape, and flow patterns:
Aneurysm size, measure fundus diameter: small 7-10 mm, medium 11-15 mm, large 16-25 mm, giant >25 mm.
Aneurysm neck size: small <50% of fundus diameter, large >50% of fundus diameter.
Aneurysm flow volume exchange rate: time needed for blood flow to flush contrast from the aneurysm: fast rate <30 sec, medium rate 30-60 sec, slow rate >60 sec.

In vitro modeling of aneurysms using simulated clinical blood flows and blood pressures have helped grade aneurysms by their ease of treatment:
Simple aneurysm: small to medium fundus, small neck, and slow volume exchange.
Moderate aneurysm: medium to large fundus, small neck, and medium to fast volume exchange.
Complex aneurysm: medium to large fundus, large neck, medium to fast volume exchange.

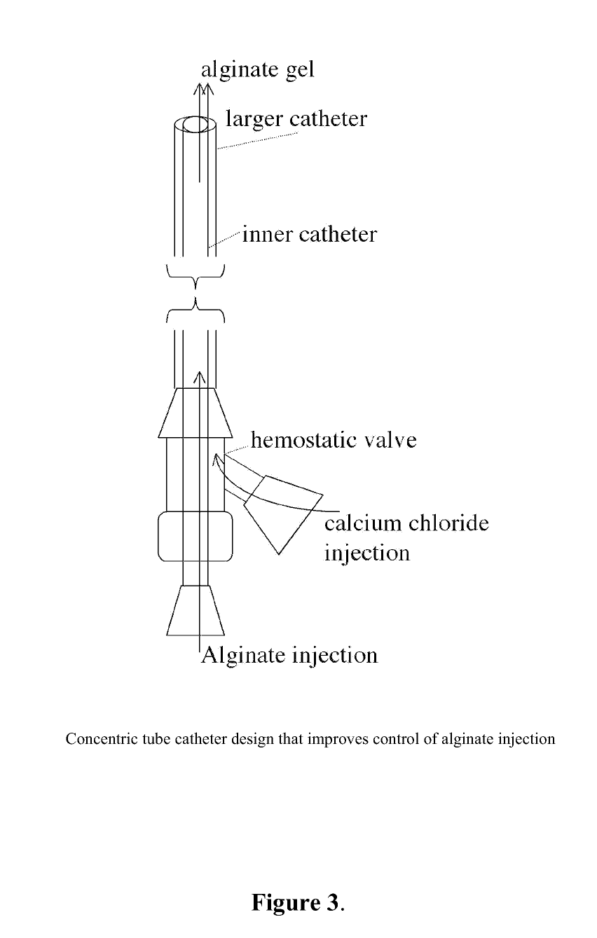

In some embodiments, the invention comprises novel alginate delivery methods which are particularly effective in low-flow and/or narrow-neck aneurysms. Thus, in some embodiments, optimal control of alginate delivery can be conducted using a concentric tube microcatheter design. The catheter consists of a single lumen microcatheter that has a second smaller-diameter catheter fed inside the first. The inner catheter is fed through a hemostatic valve or similar valve system (FIG. 3). Alginate can be injected through the inner catheter and calcium chloride injected through the side-port of the hemostatic valve, where the fluid flows inside the larger microcatheter, but outside the inner catheter. The material can be injected from either catheter site, however, alginate is more viscous than calcium chloride, and therefore there is significantly less resistance to flow when injected through the inner catheter than when injected between the inner and outer catheters. The alginate and calcium chloride mix at the exit of the catheter tips.

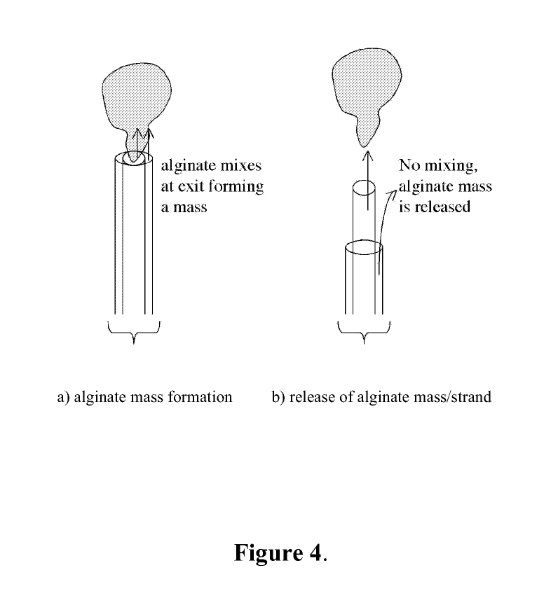

The inner catheter can be adjusted to either terminate inside the larger catheter, at the same position as the larger catheter, or outside the larger catheter. Each position has unique injection results that improve the controllability of the alginate injection and resulting alginate gel formation. Mixing inside the larger catheter creates an alginate mass that begins to form upon exit, and the gel can build upon itself to form a stable mass. Mixing at the exit of the catheter when both lumens are flush with each other creates a formable mass that can expand to completely fill a vessel defect (FIG. 4a). Releasing alginate from the inner catheter that has been placed beyond the exit of the larger catheter will minimize further mixing and thereby release any pre-formed gel from the catheter (FIG. 4b).

In accordance with embodiments of the invention, greater flow control and filling of the defect comes from alginate and calcium chloride mixing external to the catheter tip. This is accomplished by using a concentric catheter as configured in FIG. 4a. The inner and outer catheter tips are placed adjacent. The fluids are released external to the catheter and mix to a formable mass. This mass can be controlled to grow and fill the defect more completely, unlike a pre-made fiber which folds onto itself to fill the volume and thereby leaving space between the folds, much like current coil technology. Fibers are also less likely to bond to each other because the calcium chloride-alginate reaction is complete and does not consistently bond adjacent fibers together to form a mass. Mixing external to the catheter, however, forms a mass that builds upon itself to form a solid and more complete fill of the defect.

Further control is attained in some embodiments by altering the flow rates and the injection timing of the two components (alginate and calcium chloride). This technique includes, but is not limited to, uncoupled injection of the calcium chloride and alginate, asynchronous flow rates during injection, or variations in the injection start and stop times of the two components. Even synchronous or coupled injection of the two components, but using two syringes of different volumes, can be considered asynchronous because the flow rates vary for the two components. Calcium chloride and alginate flow rates can be varied during the injection and even stopped and restarted after assessing fill progress. In some embodiments, calcium chloride is always flowing whenever alginate is flowing, with a calcium chloride flow rate preferably between about 0.5× and 2× the alginate rate. As one example, without limitation, a continuous flow of calcium chloride controlled by a pump and begins before the alginate injection, then alginate is injected by hand at any flow rate deemed appropriate by the user, as long as the calcium chloride is flowing before, during and after the alginate injection occurs to guarantee the presence of calcium ions at the site of alginate delivery and therefore maximize gelation. The traditional embodiment comprises a coupled synchronous flow system that delivers exact volumes of each component at the same rate and same time. The Synchronous flow system is not recommended as a way to maximize injection control, unless the injection device can be uncoupled and controlled individually, if deemed necessary.

Asynchronous flow-rate injections allow for staged injection techniques that can be used to assess the progress of alginate filling and then continue the injection from the same catheter multiple times if needed. The staged technique also allows for addition of agents to the alginate or to the calcium chloride, including a combination of different agents that can be varied during the staged injection. Agents include but are not limited to drugs, radioactive or contrast agents, and growth factors or inhibitors.

Injection of alginate without calcium chloride is suggested only for detaching the gel mass from the catheter. This can be most easily done by pushing the inner concentric tube out past the outer tube (FIG. 4b) and injecting alginate without calcium chloride to release the mass. Unreacted alginate in the body is not an embolization concern. Unmixed calcium chloride is also not a concern for embolization or toxicity, especially at the small volumes (typically much less than 10 cc) used to gel alginate in vivo for most vessel defects.

In some embodiments, external mixing and asynchronous flow can also be accomplished with any dual-lumen catheter, with any conceivable number of lumen shapes, so long as the lumen tips are flush with each other and do not deliver the components into a mixing cannula. Rather, the components are delivered to the in vivo system and mix external to the catheter to form an occlusive mass.

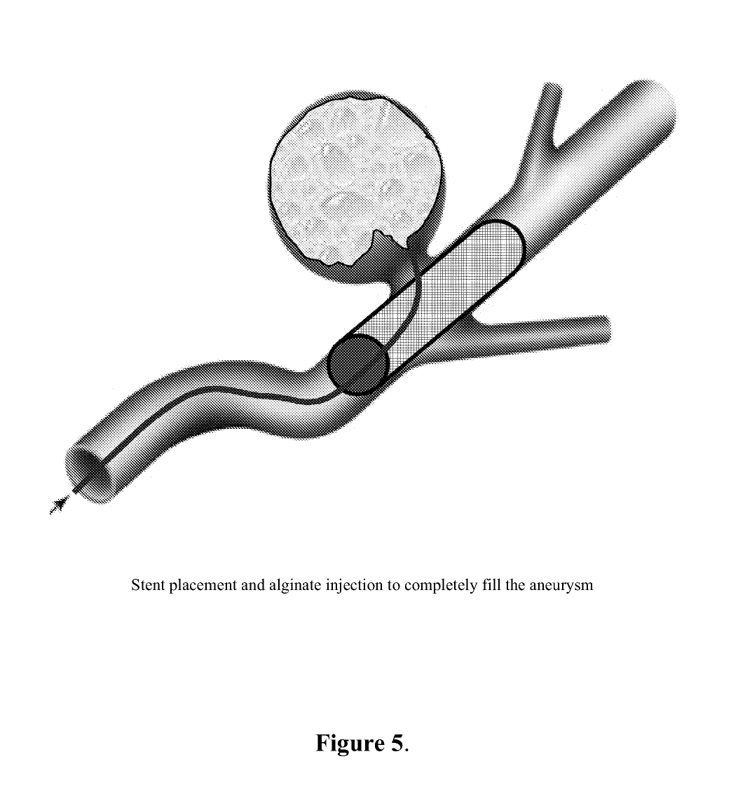

In some embodiments, without limitation, further control of the alginate gel delivery can be accomplished by first placing a stent in the parent vessel that begins proximal to the aneurysm neck and extends distal. The concentric-tube microcatheter can then be fed through the stent mesh and into the aneurysm to deliver the gel. The stent provides structural support so the alginate gel does not migrate into the parent vessel. An inflatable balloon could also be temporarily delivered across the aneurysm neck when the injection catheter is already in place to further control the alginate delivery. (FIG. 5).

Alginate Injection with Balloon Protection

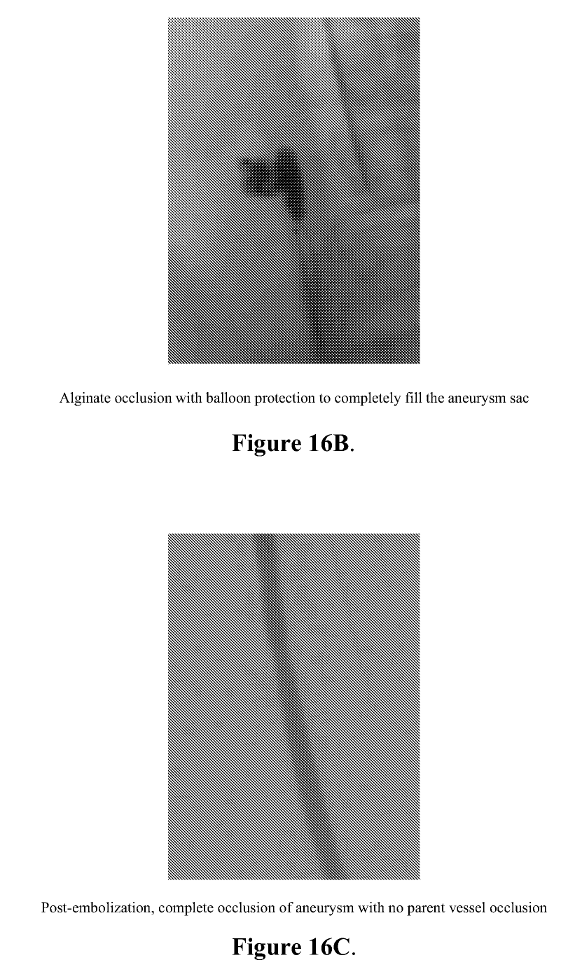

In some embodiments, without limitation, a catheter can be fed to the aneurysm and a second balloon catheter placed proximal and distal to the neck of the aneurysm and inflated to anchor the injection catheter and reduce outflow during alginate injection (i.e. FIG. 16b). After alginate delivery and gelation, the balloon is deflated and removed. The balloon can also be made of a material either non-permeable or semi-permeable to ions (such as calcium ions). With a permeable balloon, a single lumen catheter may be placed in the aneurysm, and the balloon is inflated with calcium ions. Alginate is delivered through the catheter and calcium ions permeate in from the balloon to gel the alginate. A modified system would be a single catheter with a dual lumen configuration where one lumen can be placed into an aneurysm and the second attached to a semi-permeable balloon system. The alginate and ions is delivered as stated previously, except that the catheter system is combined into one instead of two separate catheters. Balloon and catheter combination injections could be used with continuous or staged injection techniques.

### Alginate and Unmodified Coil Treatment In Vitro

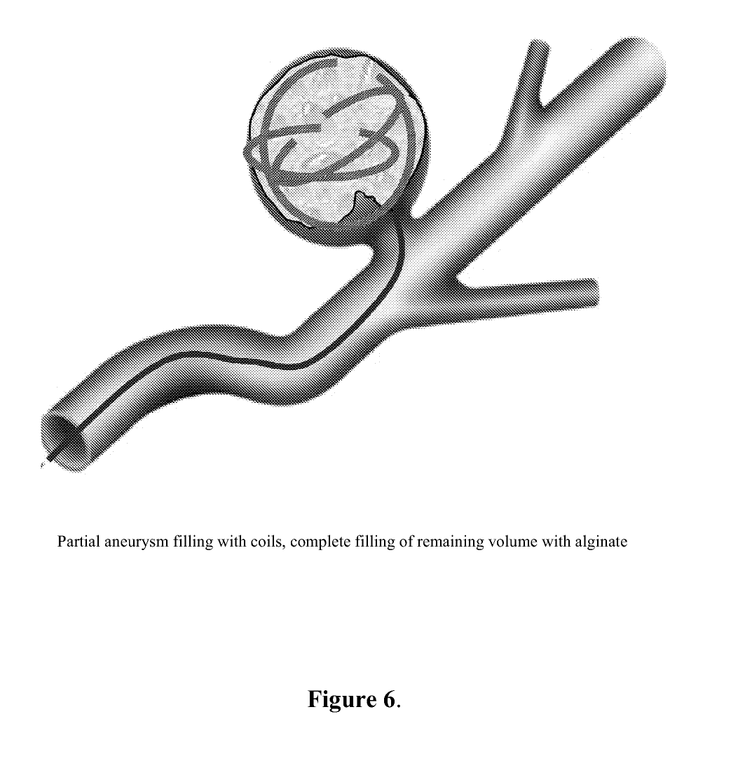

Without limitation, the invention comprises the use of alginate and unmodified coils. Our in vitro studies show that placement of coils in high flow and/or large neck aneurysms can provide structure and disrupt the blood flow effects, increasing the delivery control of alginate into the remaining aneurysm space and decreasing potential outflow into the blood stream (FIG. 6). For further protection, this method may also be combined with stent and/or balloon placement.

Our mechanical stability tests on ALGEL occlusion samples removed from in vitro aneurysm models showed that ALGEL has a mechanical stability (measured by complex modulus) that is approximately 8× higher than typical in vivo aneurysm shears. Data shows that ALGEL alone can effectively occlude small-neck, low-flow aneurysms. However, wide-neck, high-flow aneurysms require assist devices to reduce potential outflow. ALGEL combined with coils is an example of an effective treatment solution for these aneurysms.

Modified Coils Combined with Alginate Injection

Other embodiments comprise use of modified coils combined with alginate injection. Modified coil surfaces accelerate the bioactive response for tissue growth to heal an aneurysm. However, the inability to completely fill an aneurysm with coils only is a limiting factor to successful aneurysm healing. Rather, the coils of the present invention comprise a base structural component and alginate as a non-adhesive, bioactive, and tissue-like filling material that enhances occlusion stability.

Our studies show that alginate induces a positive bioactive response that promotes tissue growth. In one embodiment, without limitation, coils are impregnated with calcium ions in conjunction with alginate injection. The coils provide a structural matrix and release calcium into the fundus. Liquid alginate is then delivered to the target site, for example, from a single-lumen microcatheter, where it polymerizes in the presence of the calcium ions, creating a complete aneurysm fundus occlusion.

In one embodiment, without limitation, the invention is comprised of coil surface modification by the following steps:
1. Prepare a Type I collagen mixed with 20% calcium chloride (ionic diffusion)
2. Place the coils in collagen-calcium solution and dry to physically attach coating to the coil;
3. Ion implant the surface coating to the coil at the molecular level, increasing shear resistance; and
4. Test coil deliverability, alginate reactivity, and occlusion stability in vitro.

Studies known to those of ordinary skill have tested extensively the tissue response of surface coatings. For example, it is known that Type I collagen fibronectin induces a bioactive response that increases endothelial cell migration and proliferation on aneurysm coils. In some embodiments, these materials are mixed with 20% calcium chloride to form a coil coating that will be tested for ion diffusion and bioactivity. The coating is applied by immersing the coils in solution for 1 hour at 37′C, allowing collagen polymer arrangement on the coil surface. The coils are then air-dried in a sterile laminar hood for 1 hour.

Some studies show that dried coatings alone cannot resist the shear stress induced by catheter delivery and the shear effects of blood flow. Therefore, in some embodiments, the coating is ion implanted to the coil surface. Ion implantation has shown to increase wear, reduce corrosion (hip joints), and improve blood compatibility of a material without affecting its mechanical properties. Ion implantation of the coil coatings creates a physicochemical surface modification. Ne+ ions are accelerated and bombard the coated coil (dose of 1×10¹⁵ ions/cm² at 150 keV, other ions, such as He+, and higher energies, such as 500 KeV, can also be used to obtain similar doses). The ions form a crater-like coil surface, embedding the coating into the coil. The coils are then delivered to an aneurysm where the calcium ions are released from the embedded protein coating. The injection of alginate fills the remaining aneurysm volume and thereby isolates the aneurysm defect from the normal blood flow path. The coil placement and alginate injection can also be further protected with the use of a stent and/or balloon placed across the neck of the aneurysm during alginate injection.

Modified Coils with Alginate Coatings

Some embodiments of the invention comprise modified coils with alginate coatings. Ion releasing coils supplemented with alginate delivery can be directly compared to modified coils that contain alginate coatings. Because it is a hydrogel, alginate can be dried and rapidly rehydrated in a variety of liquid environments (such as blood). Thus, in some embodiments, the coil and alginate may be delivered as one unit. This approach has the advantage of reducing the coil treatment to one step. A perceived disadvantage, however, is the need for multiple coil insertions to completely fill the aneurysm fundus. Since coils typically only fill 25% to 33% of an aneurysm volume, the alginate hydrogel coating will have to swell and fill the remaining space. Modified alginate coated coils have been tested in vitro to determine aneurysm filling potential and coil expansion properties. Alginate is over 95% water, therefore the potential volume expansion can be significant and is worth investigating and characterizing. The creation of an alginate coating follows a similar procedure as described above. The coating procedure is summarized below:
1. Mix 1.75% alginate solution in water
2. Coil coating stage 1: dip the coils in the alginate solution, then dip in a 10% calcium chloride solution
3. Dry alginate-coated coil to create a physical attachment to the coil
4. Coil coating stage 2: ion implant the alginate coating to the coil at the molecular level
5. Test coil deliverability, alginate reactivity, and occlusion stability in vitro
6. Test coil deliverability, alginate reactivity, occlusion stability, and bioactivity in vivo

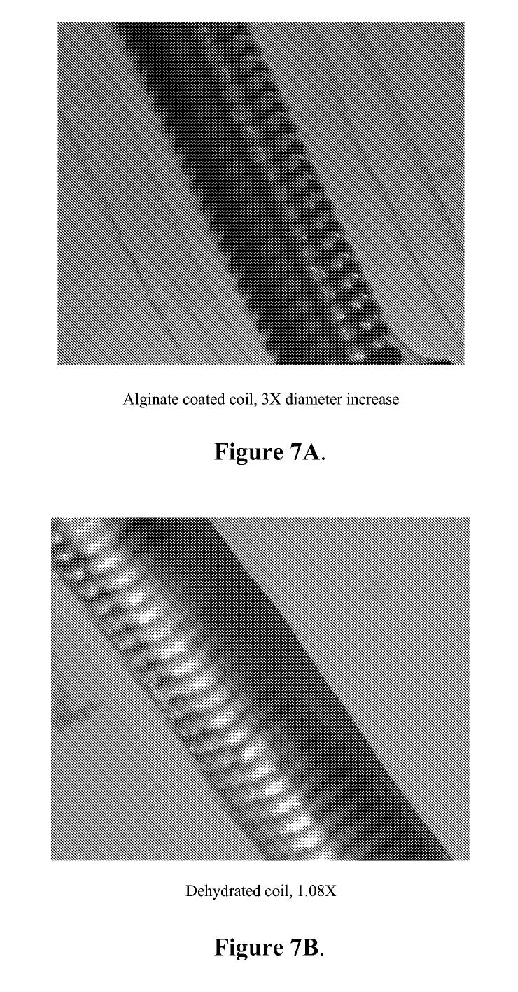

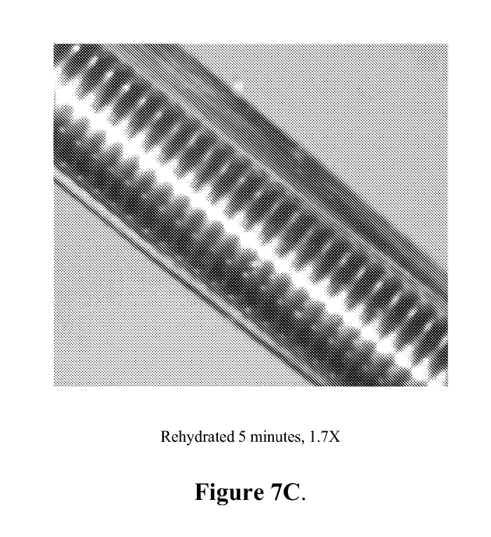

ALGEL coating of coils can improve the filling of aneurysms to attain a complete occlusion. Three coatings of ALGEL increase the coil diameter 3× (FIG. 7a), yet when dehydrated, the modified coil shrinks to nearly its original diameter, with only a 1.08× diameter increase (FIG. 7b). Then after 5 minutes back in a liquid environment, the diameter swells to 1.7× (FIG. 7c), and after 1 hour, the diameter reaches 2.7×, a regain of 90% of the original coating diameter.

These modified coils can add an additional 8-10× increase in aneurysm volume filling to maximize effective occlusion, yet can be dehydrated to near the original diameter to facilitate delivery through conventional coil delivery catheters. Modified alginate coils can also be prepared by placing a conformal coating of liquid alginate (not reacted with calcium chloride) on the coil and dehydrating the layer. The conformal coating and dehydration process can be repeated multiple times to create a coating of desired thickness. These coils could then be placed in the aneurysm, then calcium ions added either by a catheter or in combination with calcium eluding coils, as described herein.

### ADDITIONAL EXAMPLES

### Example 1

### Alginate Biocompatibility

The short- and long-term tissue reactivity was tested by injecting calcium alginate into the fat capsule surrounding the kidney of 32 rats weighing 300±50 g each. The rats were anesthetized with a ketamine cocktail (50 mg ketamine, 5 mg Xylazine, 1 mg PromAce) dose of 0.5 to 1 ml per animal. A 3 cm incision was made on the left side of the abdomen. The fat capsule around the left kidney was isolated. A pocket was made in the capsule, next to the kidney, and approximately 0.5 ml of alginate and 0.68 M CaCl₂.2H₂O, at a 1:1 volume ratio, was injected and polymerized. Each of the four polymer types was injected into the kidney of two separate rats to determine the significance of the tissue reaction during a set time period (total of 8 rats per time period). The second kidney of each rat was untouched and served as a control. Separate groups of 8 rats were sacrificed after 1 day, 1 week, 3 weeks, and 9 weeks, a total of 32 rats for the entire study. Both kidneys were harvested from each rat. Tissue reactivity was first classified by visual inspection. Polymer encapsulation, organ and tissue adhesion, and tissue necrosis are strong indicators of polymer incompatibility. Visual severity classification was adopted and modified from a nonspecific, acute ASTM standard test of polymer-tissue interaction and irritation, which consists of ranking the reactivity of the kidney and surrounding tissue on a scale of 0 to 4; 0 to 1 being little or no reaction, adhesion, or encapsulation and 4 being major adhesion, encapsulation, and/or tissue necrosis.

Crude alginate exhibits significantly higher reactivity than purified alginates, and high M acid gels induce a faster immune response than high G acid gels (Table I). Overall reactivity of crude alginate is consistently high (severity of 3 to 4) independent of acid content. Purified alginate exhibits a significantly lower immune response. The overall reactivity remains consistent between the two alginic acid concentrations (severity of 1 to 2), and the high M content alginate again exhibits a faster immune response.

TABLE I
Visual severity averages and standard deviations of polymer reactivity

| Implant | Polymer type | | | | | | | |
|---|---|---|---|---|---|---|---|---|
| Time (days) | CHM | std. dev. | CHG | std. dev. | PHM | std. dev. | PHG | std. dev. |
| 1 | 3.0 | 1.41 | 1.5 | 0.71 | 1.0 | 0.00 | 1.0 | 0.00 |
| 7 | 3.5 | 0.71 | 2.0 | 0.00 | 2.0 | 0.00 | 1.0 | 0.00 |
| 21 | 4.0 | 0.00 | 3.5 | 0.71 | 2.0 | 0.00 | 2.0 | 0.00 |
| 63 | 3.0 | 0.00 | 3.0 | 1.41 | 1.5 | 0.71 | 1.5 | 0.71 |

The studies were expanded to determine the effect of alginate structure and purity on the resulting mechanical strength and biocompatibility. It was found that alginates with a high Guluronic acid content (G/M ratio>60/40) had optimal strength, polymer yield, and biocompatibility.

### Example 2

### Alginate Molecular Weight Characterization

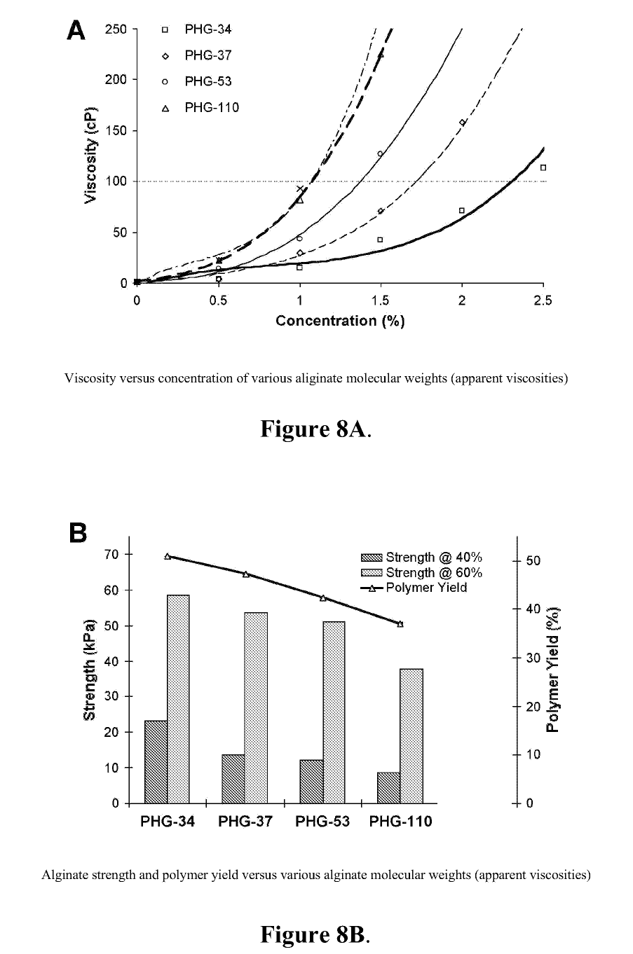

Reacted alginate molecular chain length is often referred to by the alginate's apparent viscosity (in mPas) and molecular weight (MW in g/mol). The apparent viscosity of unreacted alginate is determined by creating a 1.0 wt % solution of alginate dissolved in water and measuring its viscosity at 20° C. The apparent viscosity is proportional to the molecular weight of the alginate. Molecular weight can be measured by size exclusion chromatography with multi-angle laser light scatter detection analysis. Purified, high G acid content alginates (PHG) come in various molecular weights, which can affect the usable concentration and final viscosity of the liquid alginate in solution. Various PHG alginates were tested in vitro for mechanical stability and polymer yield based on final viscosity:
PHG alginate, apparent viscosity of 34 mPas, MW of 78,000 g/mol, G/M of 68/32
PHG alginate, apparent viscosity of 37 mPas, MW of 87,000 g/mol, G/M of 68/32
PHG alginate, apparent viscosity of 53 mPas, MW of 110,000 g/mol, G/M of 68/32
PHG alginate, apparent viscosity of 110 mPas, MW of 155,000 g/mol, G/M of 68/32.
   A range of alginate concentrations were tested for mechanical stability, and the strengths of specific viscosities of alginate were interpolated from the data set. The data was graphed and fitted with trend lines to predict compressive strength versus alginate concentration, compressive strength versus viscosity, and polymer yield versus alginate concentration. Next alginate injection viscosity was also graphed and fitted with trend lines to predict injection viscosity versus alginate concentration [FIG. 8(a)].

The resulting trend line equations were used to interpolate alginate strengths and alginate polymer yield of each alginate type at an injection viscosity of 100 cP. The results were graphed in FIG. 8(b). Interpolated data shows the trend of alginate strength and polymer yield as a function of apparent viscosity. The original, non-heat treated 34 mPas alginate has the highest strength and yield. The non-heat treated 110 mPas alginate has 60% of the strength and 75% of the polymer yield of 34 mPas alginate. However, alginates with smaller apparent viscosities that approach 34 mPas (lower molecular weights) have increased polymer yield and polymer strengths that increase respectively, approaching the mechanical characteristics of 34 mPas alginate.

Results show that alginate gels made from lower molecular weight liquid alginates are more stable than those made from long chain length alginates. Alginates with lower molecular weight can be mixed at higher concentrations than high molecular alginates to attain the same injection viscosity. The resulting low molecular weight alginate solution has a 20 to 40% greater mechanical stability of and a 5 to 10% higher polymer yield than a high molecular weight alginate solution with the same viscosity. Alginates of nearly any molecular weight range can be used (typical alginate MW range: 65,000 g/mol to 200,000 g/mol), however results show that a molecular weight range from 65,000 to 90,000 has optimal maximum strength and polymer yield.

### Example 3

### In Vitro Aneurysm Models

ALGEL occlusion studies were performed with an in vitro aneurysm models made from glass tubes and then from models cast into flexible polymer resins. The vessel models simulate the accurate vessel sizes and aneurysm sizes that form on the carotid (C) vessel, the middle cerebral (MC) branch, and the anterior cerebral (AC) branch (FIG. 3). The model allowed for endovascular embolization treatments to be tested in a simulated surgical environment. The resulting occlusions could be subjected to pulsatile flows and pressures for up to two weeks. The ALGEL samples were then removed from the model post-embolization and further analyzed for occlusion effectiveness and mechanical stability.

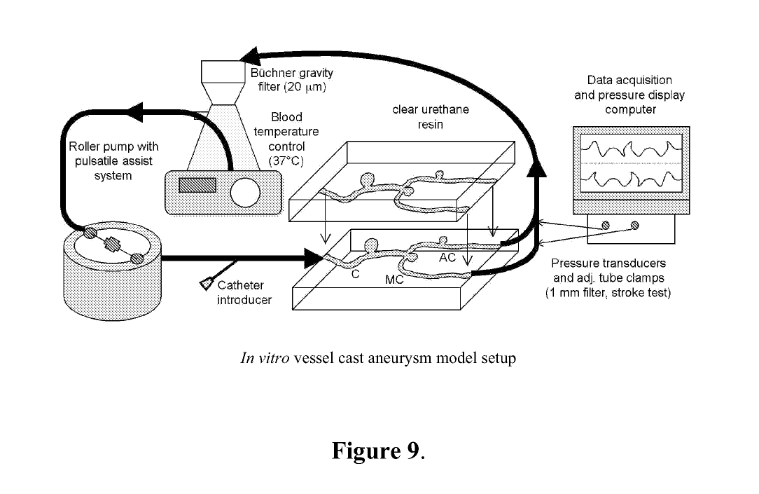

The model consisted of a pulsatile pump to simulate systolic-diastolic flow and pressure effects (200 ml/min, 160-80 mmHg). Artificial blood was used to accurately simulate viscosity, ionic content, and protein content. The artificial blood was made with 12 wt % Dextran (70,000 MW) dissolved in Ringers solution. Adjustable tubing clamps with pressure transducers were used to regulate blood flow pressure and capture large downstream particles that may occur during an over-injection. A Büchner funnel and 20 μm filter paper were used to capture any potential small particles that may pass through the transducers. Aneurysm vessels (8 mm-20 mm fundus, small neck: 3-6 mm, wide neck: 7-14 mm) were molded into flexible and compliant resins (CF50 Urethane) in two form-fitting pieces that clamped together to form the flow system (FIG. 9). The model was catheterized through the flexible tubing to simulate femoral access to the carotid artery pathway. Neuroradiological devices and catheters were fed into position using a fluoroscope imaging system. In one embodiment of the invention, without limitation, in vivo pressures and flow rates were simulated in a model of a bifurcation aneurysm and two side-wall aneurysms. Pre-embolization model flow was determined with the fluoroscope (FIG. 9). After ALGEL injection, the model was opened to access embolic material and remove it for further analysis.

The aneurysm components of the model were occluded in two ways: 1) ALGEL injection only, and 2) a combination of partial aneurysm coiling, followed by ALGEL injection.

ALGEL injection into small-neck aneurysms was expected to provide complete occlusion. However, giant aneurysms and wide-neck aneurysms have significantly different flow properties, and therefore a greater potential for ALGEL flow downstream without the use of preventative measures. Therefore, a base of 2-3 coils was placed in the wide-neck aneurysms (<5% volume filled). The coils served as a matrix structure and ALGEL was then injected to fill the remaining space.

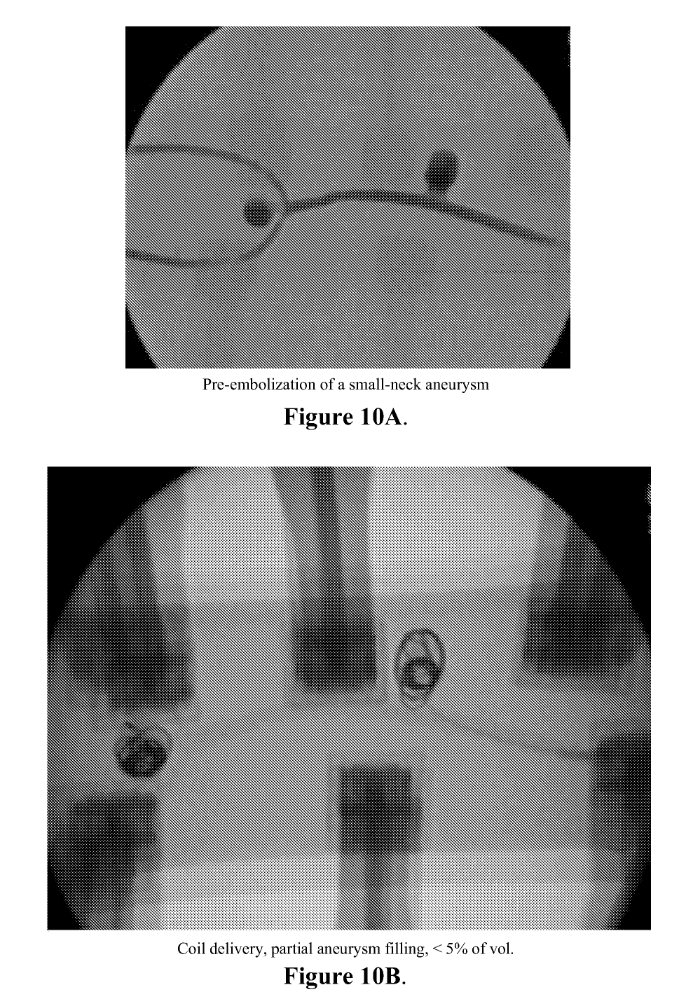

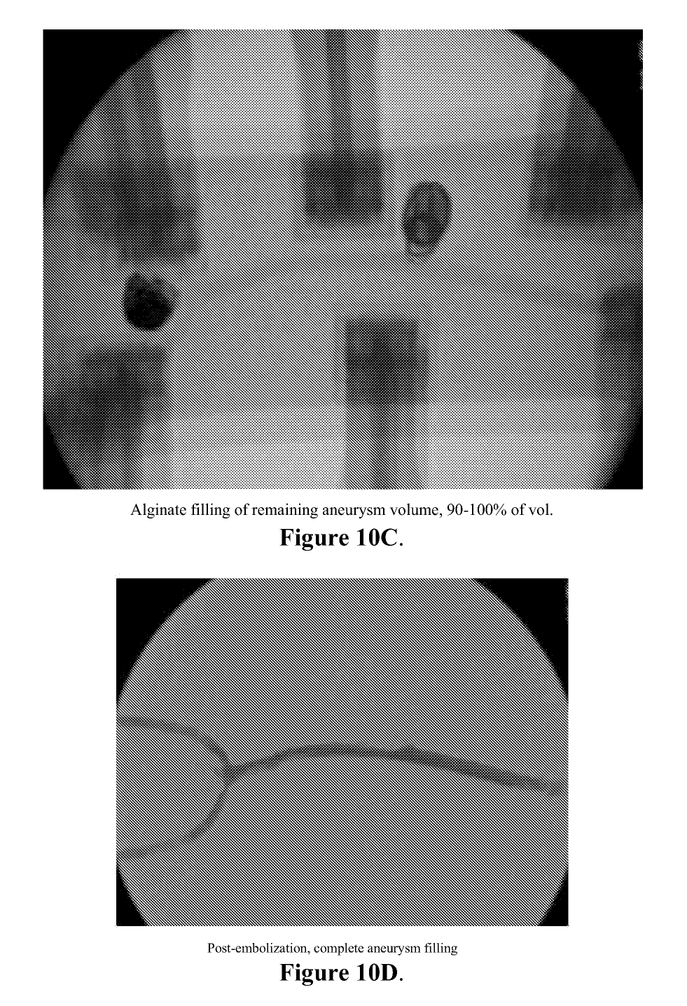

Pre-embolization angiograms were taken to image the flow into and out of the small-neck aneurysm model (FIG. 10a). Commercial endovascular coils (Detach-18, Cook Inc.) were delivered to the aneurysm to form a structural matrix and stop turbulent flow in the aneurysm fundus (max. of three coils used, 5% vol. occluded, FIG. 10b). The injectable ALGEL mixture, (1.6 wt % 37 mPas PHG alginate mixed with 50% Conray in water and 0.25 g tantalum per 1 ml of ALGEL) was tested extensively and optimized to provide maximum visualization in vitro and in vivo, as well as low viscosity in liquid from and high mechanical strength in gel form. A 3F double lumen microcatheter (Target Therapeutics, Fremont Calif.) was inserted into the inlet stream and fed to the aneurysm utilizing angiographic imaging. The ALGEL was delivered along with the alginate re component, calcium chloride, to occlude the aneurysm fundus (FIG. 10c). Aneurysm filling with coils and alginate created a 90% to 100% occlusion of the aneurysm. Analyses of fluoroscope image density pre- and post-occlusion were compared to assess occlusion effectiveness and identify any potential downstream occlusions. Post-occlusion angiograms showed removal of the aneurysm from the vessel flow (FIG. 10d). The models were then disconnected from the flow, and the halves were separated to access to the vessel lumens and compare visual occlusion results with radiographic images. The model was then cleaned out, the halves were re-clamped together, and the model reused for further injection experiments.

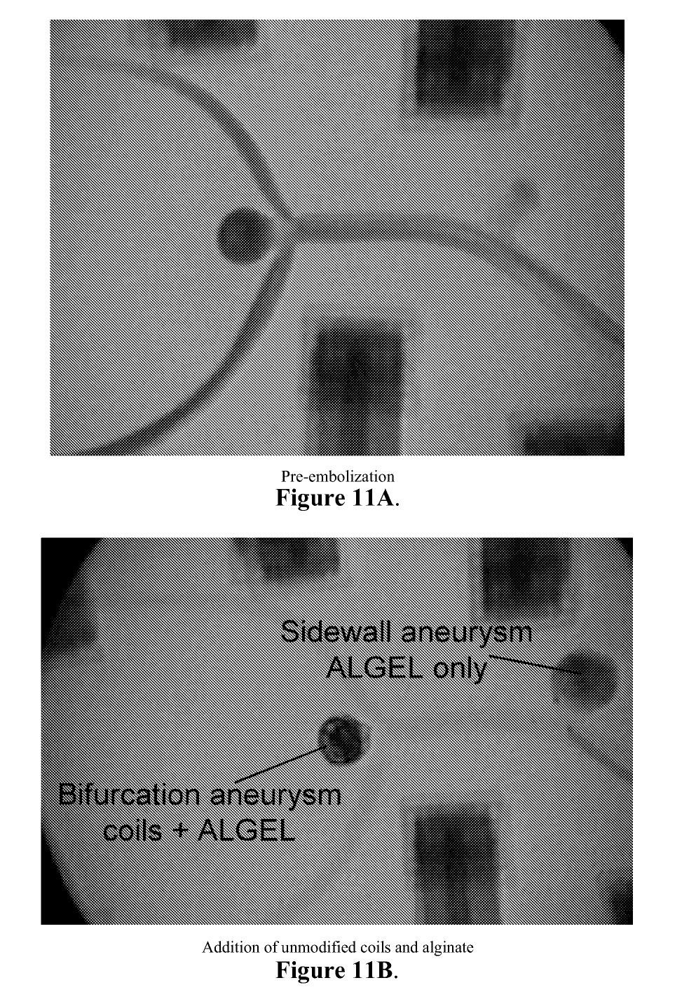

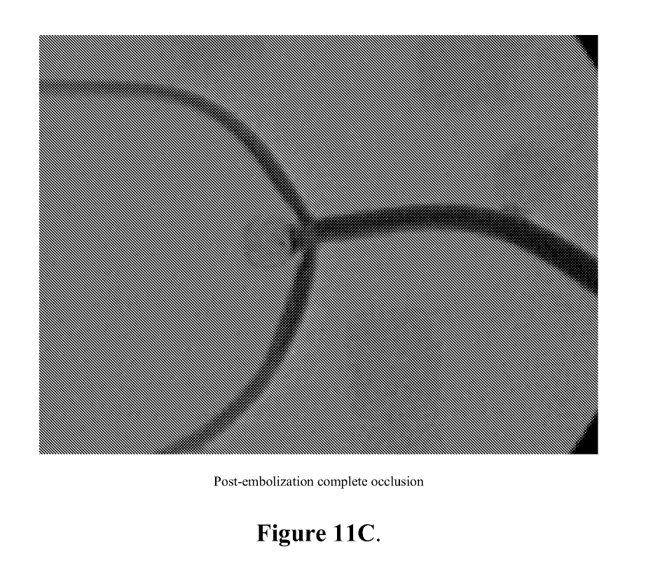

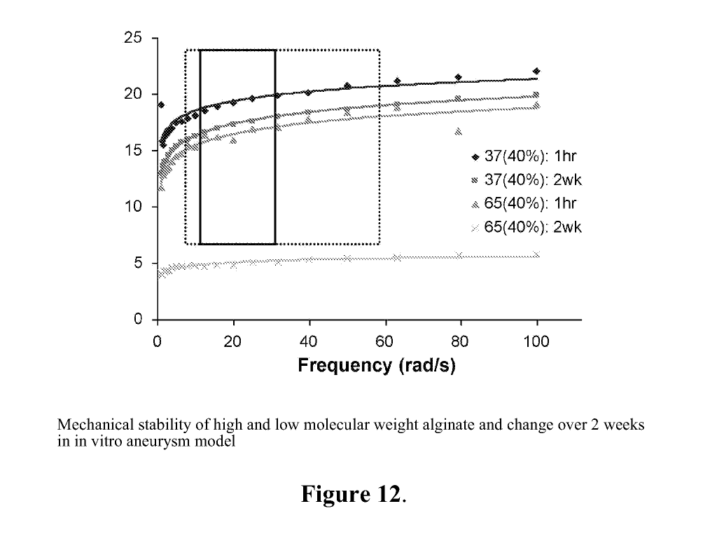

In further tests a wide-neck aneurysm model was used (FIG. 11a). A combination of up to three coils was followed by ALGEL injection into the coil matrix to occlude the high-flow, wide-neck bifurcation aneurysm (FIG. 11b). The post-embolization angiogram showed complete occlusion of the aneurysms with no downstream flow and sustained patent flow through the vessel model (FIG. 11c).

The following table summarizes the embolization treatments of the completed ALGEL occlusions and the preliminary ALGEL-coil occlusions (Table II):

TABLE II
Occlusion success

| | |
|---|---|
| # aneuysms | % controlled |

Wide-neck aneurysms

| | | |
|---|---|---|
| ALGEL only | 22 | 27 |
| Coils + ALGEL | 5 | 100 |

Small-neck aneurysms

| | | |
|---|---|---|
| ALGEL only | 15 | 80 |
| Coils + ALGEL | 3 | 100 |

ALGEL-coil test results show an enhanced occlusion technique for wide-neck aneurysms. Several aneurysm sizes were cast in flexible resins to simulate side-wall and bifurcation aneurysms in an in vitro system. First, ALGEL was delivered to small neck aneurysms from a 3F dual-lumen microcatheter. Second, a minimal number of coils were delivered to wide-neck aneurysms to form a matrix structure. ALGEL was then delivered to fill the remaining aneurysm space. ALGEL completely and effectively filled both small-neck aneurysms and, when combined with coils, completely filled wide-neck, high-flow aneurysms and eliminated outflow.

The alginate occlusions were recovered from the in vitro model tested for gel volume and mechanical stability. Volume was measured with a 5 cc syringe was prefilled with 2 cc of artificial blood and the ALGEL sample was submerged in the fluid. The volume displacement was noted as the ALGEL sample volume. The ALGEL volume was compared to the known aneurysm volume and represented as a percent filling.

Mechanical stability was tested with a rheometer (RMS-800/RDS Rheometrics Scientific) to measure complex modulus and resistance to shear at 37° C. (body temperature) and 1% strain across a frequency sweep of 1 to 500 rad/s.

Complex modulus was compared to the typical shear stress and shear frequency sweeps seen in vivo. Shear stress on an aneurysm can be estimated by the following equation:

$${τ}_{w} = \frac{Δ Pd}{4 L} \qquad ( 1 )$$

where (L) is the longitudinal width of the aneurysm neck, (d) is the internal diameter of the vessel, and (ΔP) is the systolic-diastolic change in pressure across the aneurysm neck. Stress frequency sweep for an in vivo system was estimated by converting typical blood flow velocities (ν) to radians per second (rad/s) using the ALGEL sample radius (r):

Rad/s=ν/r (2)

The calculated shear and frequency estimations for an in vivo system were compared to the actual shear resistance of the samples tested across an expansive frequency range that included the estimated in vivo frequency range (Table III).

TABLE III
Comparison of calculated in vivo shears ranges to actual
in vitro shear resistance of alginate

| | freq. | calc. in vivo | actual in vitro | strength factor |
|---|---|---|---|---|
| | (rad/s) | shear (kPa) | shear (kPa) | actual/calc. |
| max | 63.0 | 7.1 | 21.1 | 3.0 |
| typical | 25.1 | 1.1 | 19.5 | 18.2 |
| min | 7.9 | 0.1 | 17.8 | 161.5 |

Results of the mechanical stability and fatigue resistance results showed that low molecular weight alginates (65,000-90,000 g/mol) have superior short- and long-term fatigue resistance. High molecular weight alginates had good initial stability, but degraded in strength over time (tested after 2 weeks in simulated in vivo conditions—FIG. 12).

Alginate gel volume decreases over time due to liquid loss of the gel from constant in vivo pressures, but the % fill of the aneurysm remains between 60% and 90% (Table IV).

TABLE IV
Change in alginate % filling of aneurysm over time

| comparison | 95% conf. | p value | | vol % | st. dev. |
|---|---|---|---|---|---|
| 37-1 hr = 37-2 wk | yes | 0.753 | 37-1 hr | 80 | 5.0 |
| 37-1 hr = 65-1 hr | yes | 0.630 | 37-2 wk | 63 | 9.1 |
| 37-2 wk = 65-2 wk | no | 0.029 | 65-1 hr | 65 | 2.4 |
| 65-1 hr = 65-2 wk | no | 0.002 | 65-2 wk | 65 | 8.5 |

Mechanical stability results show that optimized alginate (37 mPas PHG alginate) has a shear resistance that is up to 20× greater than the shear effects seen in the human vascular system. Low molecular weight alginates (20-40 mPas, or 65,000-90,000 g/mol) have superior short- and long-term fatigue resistance as tested for up to two weeks.

### Example 3

### In Vivo AVM and Aneurysm Studies

Studies with embolizing in vitro aneurysm swine models with alginate show that the alginate completely filled and occluded the aneurysm fundus (FIG. 10a-d & FIG. 11a-c).

In other embodiments of the inventions, in vivo vessel models were created in the neck of swine, based on swine models of an AVM lesion known to those of ordinary skill in the art. The results showed that ALGEL could be precisely visualized with modern fluoroscope equipment and focally delivered to precise areas of the vessel model, resulting in complete occlusion with no distal embolization.

Swine studies also resulted in a new chronic swine model that could be used to determine an endovascular gel's long-term mechanical stability, biocompatibility, and bioactive tissue growth response. The chronic model has been used extensively to focally deliver ALGEL without the concern of particulate flow downstream. Current studies show that the ALGEL delivery and reaction properties downstream particulates have been verified in chronic animals survived for up to 6 months. Effective ALGEL occlusion, biodompatibility and a lack of downstream particulates were verified in chronic animals survived for up to six months.

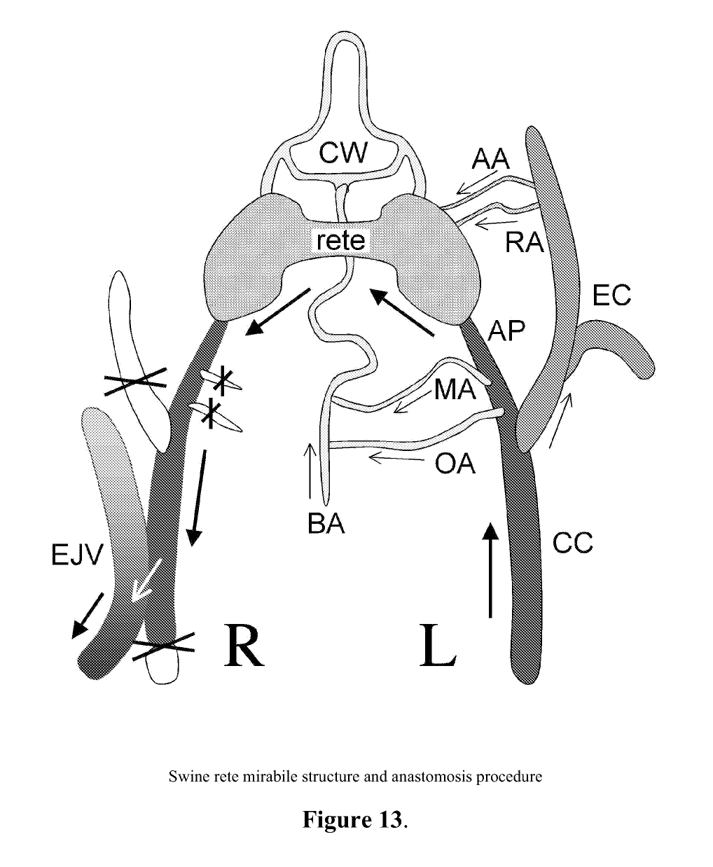

The swine RM is a network of vessels found in the base of the skull (FIG. 13). The RM is fed from both the left and right common carotid (CC) arteries. The CCs branch just before the base of the skull into the external carotid arteries (EC) and the ascending pharyngeal arteries (AP). The left and right AP directly feed the inferior portion of the RM. The superior portion of the RM connects to the circle of Willis (CW), supplementing blood flow from the basilar artery (BA). The superior RM is also connected to the EC by the ramus anastomoticus (RA) and the arteria anastomotica (AA). Smaller vessels branch from the AP, the occipital arterial branch (OA) and the muscular arterial branch (MA), and bypass the RM. Blood flow exits the model from the external jugular vein (EJV).

A 15 cm incision is made on the right side of the neck, parallel to the sternocleidomastiod muscle, to the base of the skull. A 5 cm segment of the EJV and the CC is dissected, isolated, and cleaned of adventitia. A 2 cm longitudinal incision is made in the CC segment and the adjacent EJV. The vessel lumina are washed of blood with heparinized saline. The posterior edges of the incisions are approximated and anastomosed with continuous 6-0 prolene suture, and then the anterior edges are anastomosed to complete the fistula.

The resulting blood flow crosses at the anastomosis, exiting through the EJV. The CC, proximal to the anastomosis, is ligated and coagulated to prevent flow from the carotid into the anastomosis. The CC, distal to the anastomosis, is followed to its bifurcation into the EC and AP near the base of the skull. The EC is then ligated at its origin with 6-0 prolene and coagulated with bipolar cautery. The OA and the MA of the AP are the secondary flow paths that bypass the RM, therefore these branches are also ligated or coagulated. The result is a blood flow loop, with the left CC and AP acting as arterial feeders, the rete mirabile becomes an AVM mass (nidus) and the right AP, CC, and EJV become the venous drainage system (FIG. 13).

The in vivo swine aneurysm model is a well-documented procedure for creating aneurysms and testing occlusion materials, such as coils, in a chronic setting. A 10 cm incision is made on the right side neck. The common carotid artery (CCA), internal carotid artery (ICA), and the external carotid artery (ECA), and the carotid bifurcation are exposed and the external jugular vein is exposed (EJV). All vessel surgery and aneurysm construction is performed using a surgical microscope by a neurosurgeon or an expert researcher. After exposing a sufficient length of EJV, it is ligated at the ends. A 2 cm section of EJV is then removed and placed in saline. The removed EJV is then cut into a smaller section to create the aneurysm fundus. The distal lumen of the vessel is cut and the vessel wall is sewn shut to form the spherical fundus. The aneurysm fundus created will have an elliptical shape with a major diameter of 8 mm and a minor diameter of 6 mm. The neck diameter will be approximately 4 mm. After clamping the carotid vessels, circular side wall cuts are made along the length of the exposed common carotid (usually the ICA and ECA may also be used). The proximal open end of the modified EJV segment is then sewed in an end to side fashion onto the side wall of the carotid vessel, creating a saccular aneurysm pouch. By varying the length of section of EJV and the size of the carotid vessel opening, aneurysms with varying neck sizes and fundus sizes can be constructed.

Long-term embolization studies of alginate have been conducted on 13 AVM swine models and 3 aneurysm models. Of the AVM models, 4 were survived 1 week, 3 for 1 month, and 6 for 6 months. The 3 aneurysm models were survived for 1 month. All animals were embolized with 1.6 wt % 37 mPas (87,000 g/mol) PHG alginate dissolved in 50% Conray and water, and mixed with 0.25 g tantalum per 1 ml of ALGEL solution. The ALGEL injections were conducted with 150 cm, 3F prototype double lumen or concentric-tube microcatheters (Target Therapeutics, Fremont, Calif.). The double-lumen microcatheter design allowed for the simultaneous injection of liquid ALGEL and the reactive component, calcium chloride, separately until mixing and polymerizing upon exit from the microcatheter tip. Treatment involved partial occlusion of the inferior portion of the left RM and total occlusion of the AP vessel in the AVM models, and complete occlusion of the fundus sac in the aneurysm models. Acute aneurysm model injections were conducted with the following protective devices: stent, coil(s), balloon, stent and coil(s), stent and balloon, coil(s) and balloon. All 3 survival aneurysm models were embolized with alginate and a balloon.

Fluoroscopy was performed with an OEC 9800 Series Super-C fluoroscope with image digitization on an OEC 1 k×1 k workstation (OEC Medical Systems Inc., Salt Lake City, Utah). The double lumen catheter/concentric catheter injection was introduced through a 6F guide catheter, to the entrance of the RM (for the aneurysm model, an 8F guide catheter was used to accommodate the introduction of the injection and balloon catheters). Purified ALGEL (37 mPas (87,000 g/mol) PHG, heat-treated batch # 411-256-06, Pronova Biomedical, Oslo, Norway) and its reactive component, 0.68 M calcium chloride anhydrous (CaCl₂), were then delivered to the left RM. The more viscous ALGEL component (approx. viscosity of 130 cP) was injected from a 3 cc syringe at 1 to 1.2 ml/min with a high-pressure syringe pump (High Pressure ‘44’, Harvard Apparatus, Boston, Mass.). Injection volumes ranged from 0.2 to 0.6 ml. The reactive component, CaCl₂, was injected simultaneously through the adjacent catheter lumen with a 10 cc syringe at 0.75 to 0.9 ml/min (previous studies showed that the optimal reactive component injection rate was 75% of the ALGEL injection rate [3,4]) with a standard syringe pump (PHD 2200, Harvard Apparatus, Boston, Mass.).

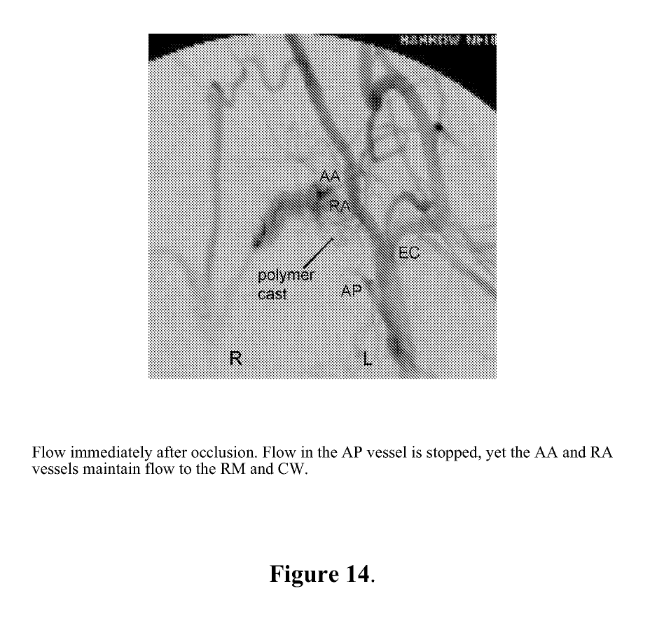

The partial occlusion technique required two or more injections of approximately 0.1 to 0.2 ml of ALGEL. The angiogram showed that the first injection flowed into the inferior portion of the RM and occluded a section of the lower vessels. The remaining injections, done within five minutes of the first and with the same microcatheter, flowed into the remaining open vessels at the inferior entrance to the RM. An angiogram verified that flow to the inferior half of the left RM was blocked, yet flow to the superior portion of the RM from the RA and AA was maintained (FIG. 14).

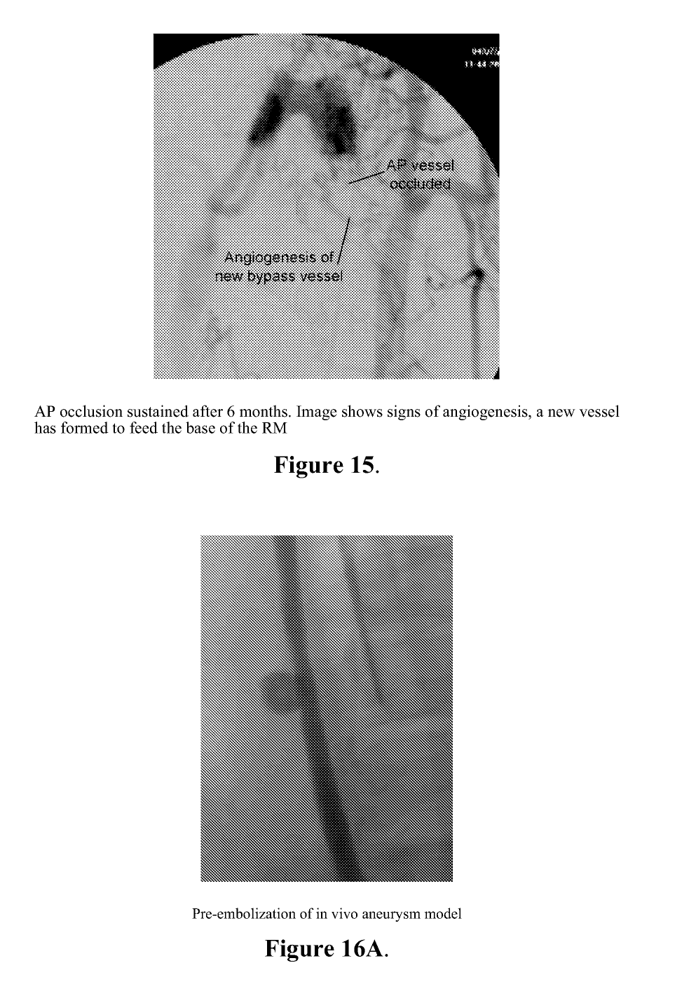

All nine swine recovered from the partial embolization procedure and were survived: three for one month and six for six months post-embolization. All nine swine showed no signs of neurological deterioration or abnormal behavior. A final angiogram, done immediately prior to sacrifice of the animals, showed that the left AP vessel remained occluded during the six-month survival. The superior RM and the CW remained patent in all nine chronic animals. The angiogram showed marked dilation of the feeding vessels (basilar, AA and RA vessels) as well as recruitment of new vessels to compensate for flow lost to the occluded AP vessel (FIG. 15).

Fluoroscopic imaging during the aneurysm embolization procedure showed vessel flow and aneurysm filling pre-embolization (FIG. 16a). The alginate was then injected to fill the aneurysm sac with protection of a balloon (FIG. 16b). The balloon was removed and vessel flow was imaged post-embolization. No signs of the aneurysm could be seen, verifying complete aneurysm occlusion (FIG. 16c).

The survival aneurysm model occlusions resulted in 90-100% occlusion of the aneurysm sac, and all 3 survival animals recovered with no signs of neurological deterioration or stroke.

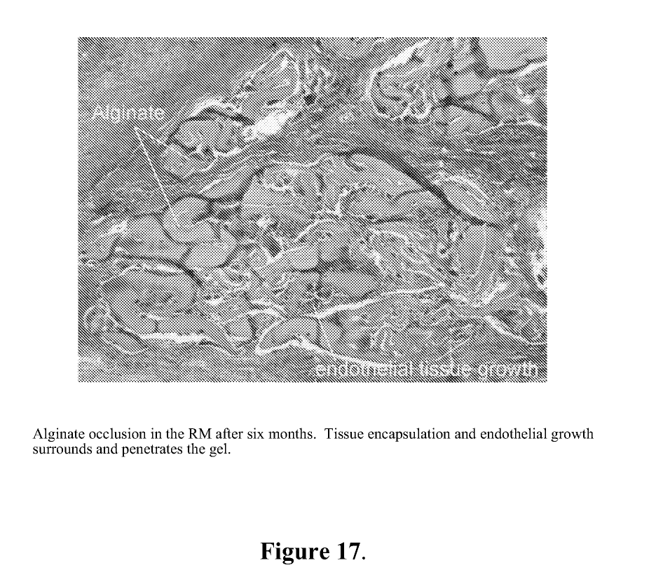

Histology on the AVM model tissue verified that ALGEL was concentrated in the inferior portion of the RM, as seen by angiographic tracking of the ALGEL injection into the left RM. No signs of ALGEL were found in the sectioned CW histology slides. Histology of the RM occlusion showed endothelial growth around the ALGEL. The vessel walls appeared intact, with no signs of tissue damage. The ALGEL underwent encapsulation that stabilized the occlusion long-term (FIG. 17).

1-month follow-up angiograms on the 3 occluded aneurysm swine models showed that all three aneurysm models remained occluded and the parent vessel remained open. No evidence of alginate degradation or downstream propagation of the occlusion material was seen. No evidence of an abnormal immune response was seen, as determined by the parent vessel remaining patent. A controlled bioactive response appeared to seal the aneursym neck, effectively removing the aneurysm from the normal flow in the parent vessel. No overgrowth of abnormal tissue was seen at the aneurysm site, therefore no flow impedement or blockage was seen in the adjacent parent vessel.

ALGEL is non-adhesive and catheter retention was not an issue. ALGEL appears to promote a positive bioactive response, and tissue growth that strengthens the polymer plug and serves as a permanent occlusion of the AVM and aneurysm area.

While the present invention has been particularly shown and described with reference to the foregoing preferred and alternative embodiments, it should be understood by those skilled in the art that various alternatives to the embodiments of the invention described herein may be employed in practicing the invention without departing from the spirit and scope of the invention as defined in the following claims. It is intended that the following claims define the scope of the invention and that the method and apparatus within the scope of these claims and their equivalents be covered thereby. This description of the invention should be understood to include all novel and non-obvious combinations of elements described herein, and claims may be presented in this or a later application to any novel and non-obvious combination of these elements. The foregoing embodiments are illustrative, and no single feature or element is essential to all possible combinations that may be claimed in this or a later application. Where the claims recite “a” or “a first” element of the equivalent thereof, such claims should be understood to include incorporation of one or more such elements, neither requiring nor excluding two or more such elements.

1-32. (canceled)

33. A method for forming an endovascular occlusion comprising the step of controlling injection of a purified alginate liquid and injection of a calcium chloride solution to a targeted area within a vascular system, wherein the injection of the purified alginate liquid and injection of the calcium chloride solution are at variable injection rates, either within an injection stage or across injection stages, and wherein the purified liquid alginate is of a molecular weight from 65,000 g/mol to 200,000 g/mol.
